(19) United States
(12) Patent Application Publication
(10) Pub. No.: US 2023/0136199 A1
(43) Pub. Date: May 4, 2023

(54) SYSTEMS AND METHODS FOR TREATING GI TRACT DYSBIOSIS

(71) Applicants: John H. SHADDUCK, Menlo Park, CA (US); Michael HOEY, Shoreview, MN (US)

(72) Inventors: John H. SHADDUCK, Menlo Park, CA (US); Michael HOEY, Shoreview, MN (US)

(21) Appl. No.: 17/931,480

(22) Filed: Sep. 12, 2022

Related U.S. Application Data
(60) Provisional application No. 63/263,392, filed on Nov. 2, 2021.

(51) Int. Cl. A61M 25/10; A61B 18/04; A61K 35/74

(52) U.S. Cl. CPC A61M 25/1011; A61B 18/04; A61K 35/74; A61B 2018/00577

(57) ABSTRACT

Systems and methods for the treatment or prevention of dysbiosis in the gastrointestinal tract of an individual and includes implantable devices adapted to release therapeutic or restorative microbiota to an individual's GI tract as well as ablation systems that can ablate residing microbiota before administering the restorative microbiota.

### RELATED APPLICATION

This is a non-provisional application of U.S. provisional application No. 63/263,392 filed on Nov. 2, 2021, the entirety of which is incorporated by reference herein.

### FIELD OF THE INVENTION

The present invention relates to systems and methods for the treatment or prevention of dysbiosis in the gastrointestinal tract of an individual and includes implantable devices adapted to release therapeutic or restorative microbiota to an individual's GI tract as well as ablation systems that can ablate residing microbiota before administering the restorative microbiota.

### BACKGROUND

The intestinal mucosa in a human is the largest body surface, approximately 200 m², that is exposed to the external environment. At birth, the intestines and mucosa are thought to be sterile, but after birth, a large variety of maternal and environmental microbes rapidly colonize the intestinal mucosa to form a unique gastrointestinal (GI) tract microbiota population. Over time, the microbiota stabilizes, and its content is composed of many species of microorganisms, including bacteria, yeast, and viruses. In an adult individual, the gastrointestinal microbiota comprises about 10¹⁴ bacteria, with a bacterial genome having from 200,000 to 800,000 genes per individual, i.e., 10 to 50 times the number of genes of the human genome.

Individuals may have quite similar bacterial species, but the exact microbiota composition in terms of bacterial species and proportions is, to a large extent, specific to the host. Thus, GI tract microbiota is a very diverse, complex ecosystem that is specific to each individual.

Taxonomically, the bacteria are classified according to phyla, classes, orders, families, genera, and species. While a few phyla are typically represented, these phyla may account for between 500 and 1000 discrete species in the individual's GI tract. The dominant microbial phyla are Firmicutes, Bacteroidetes, Actinobacteria, Proteobacteria, Fusobacteria, and Verrucomicrobia, with the two phyla Firmicutes and Bacteroidetes likely representing 90% of GI tract microbiota. The Firmicutes phylum is composed of more than 200 different genera, such as Lactobacillus, Bacillus, Clostridium, Enterococcus, and Ruminicoccus. Clostridium genera represent 95% of the Firmicutes phyla. Bacteroidetes consist of predominant genera such as Bacteroidetes and Prevotella. The Actinobacteria phylum is proportionally less abundant in a typical individual. Bacteria from the Lactobacillus family also may also be present in a GI tract microbiome.

Some GI tract microbiome activities are well understood, such as the ability of certain microbiota to break down carbohydrates that go undigested by the host's digestive system to provide energy in the form of short chain fatty acids and nutrition. In addition to providing energy, the complex, symbiotic microbiome ecosystem interacts within the gastrointestinal tract to provide many key metabolic end-products that are essential to the health of the host. The GI tract microbiome influences and yields a wide variety of functions, including metabolic function, immune function, gut integrity, bile and lipid metabolism, various organ functions (i.e., heart, liver, brain, etc.), and susceptibility to infections of the gastrointestinal tract. Recent investigations have found that GI tract microbiome exerts a considerable influence on human neurophysiology and mental health. Interactions between the intestinal microbiome and host regulatory systems are implicated in the development of psychiatric conditions and in the efficacy of common therapies.

It is essential for the health of an individual to maintain a stable, diverse microbiota that can return to its initial state after a change and is resistant to pathogen invasion. A rich and diverse GI tract microbiota is best adapted to withstand external threats. The GI tract microbiota represents a varying ecosystem that can be severely tested by unbalanced diets, stress, antibiotics, and diseases. Further, many pathologies and medical treatments can disrupt the microbiota, leading to GI tract dysbiosis. For example, inflammatory diseases, such as chronic intestinal inflammatory diseases, can limit intestinal microbiota diversity. Iatrogenic dysbiosis occurs when the imbalance is caused by a medical intervention or treatments, such as antibiotic treatments. A healthy host-microbiome balance is needed to optimally perform metabolic and immune functions and prevent disease development.

There is a clinical need for systems and methods for stabilizing, restoring and/or regulating the GI tract microbiome.

### SUMMARY OF THE INVENTION

The present disclosure relates to methods of treating a gastrointestinal tract dysbiosis. For example, such a method can include delivering a condensable vapor to a targeted region of a lumen of a gastrointestinal tract to ablate a luminal surface and a mucous layer of a surface of the gastrointestinal tract without ablating a submucosal layer of the gastrointestinal tract such that a microbiome carried on or in the mucous layer is ablated.

In some aspects, the techniques described herein relate to a method further including delivering a catheter to the targeted region and expanding a first occlusion balloon and a second occlusion balloon to engage a wall of the gastrointestinal tract, where the first occlusion balloon is spaced apart from the second occlusion balloon, and delivering the condensable vapor through an inflow channel of the catheter to the targeted region between the first occlusion balloon and the second occlusion balloon.

The techniques described herein can relate to a method wherein the condensable vapor undergoes a vapor-to-liquid phase change that applies ablative thermal energy to the luminal surface and mucous layer.

In additional variations, the techniques described herein relate to a method wherein a portion of a liquid condensate resulting from the vapor-to-liquid phase change flows outward from the targeted region through an outflow channel and an outlet in the catheter.

The techniques described herein can relate to a method wherein the vapor-to-liquid phase change ablates villi that are immersed in the mucous layer.

The method can further include ablating the luminal surface and mucous layer sequentially in a plurality of locations by re-positioning the catheter and occlusion balloons.

In some aspects, the techniques described herein relate to a method wherein ablating the luminal surface and mucous layer is provided over a continuous length of the GI tract of at least 20 cm.

The methods described herein can further include administering a restorative microbiota to the region of the gastrointestinal tract in which the residing microbiota was ablated.

Additional variations include methods wherein at least one sensor of at least one occlusion balloon send a signal of balloon contact with the surface of the lumen.

In some aspects, the techniques described herein relate to methods of treating a gastrointestinal tract dysbiosis, the method including: administering a restorative microbiota to the gastrointestinal tract of a patient over a selected time interval from a time-release element, wherein the restorative microbiota includes an effective amount of bacteria from two or more different taxonomic families selected from the group of Bacteroidaceae, Acintobacter, Firmacutes, Protetobacter, Lactobacillus, Fusibacter, Ruminococcaceae, Rikenellaceae. Clostridiaceae and Lachnospiraceae. The techniques described herein can relate to a method wherein the restorative microbiota includes Anaerobutyricum Soehngenii. In some aspects, the techniques described herein relate to a method wherein the restorative microbiota includes a processed human fecal composition. The restorative microbiota can include a lyophilized composition.

The methods can include a time-release element that is an implantable element attached to the gastrointestinal tract. In some aspects, the implantable element includes biodegradable materials adapted to biodegrade to release the restorative microbiota to the gastrointestinal tract. Variations of the method include the implantable element including a tethered element that is attached to a wall of the gastrointestinal tract by a clip.

In some aspects, the techniques described herein relate to a system wherein the restorative microbiota includes Anaerobutyricum Soehngenii.

In some aspects, the techniques described herein relate to a system wherein the restorative microbiota is a lyophilized material.

In some aspects, the techniques described herein relate to a catheter for treating GI tract dysbiosis of a patient, including: a handle, a flexible catheter shaft extending from the handle having a proximal end and a working end carrying at least a proximal occlusion balloon and a distal occlusion balloon wherein the proximal occlusion balloon is at least 100 cm from the proximal end of the flexible catheter shaft, and a heating mechanism capable of converting a flow of liquid media to a flow of vapor media that exits the flexible catheter shaft at an outlet located between the proximal occlusion balloon and the distal occlusion balloons wherein the vapor media has a vapor quality of at least 80% vapor, 85% vapor or 90% vapor, and wherein the flow of vapor media from the outlet delivers at least 5 cal/sec to a GI tract lumen in which the proximal occlusion balloon and the distal occlusion balloon are expanded.

Variations of the methods can include treating a gastrointestinal (GI) tract dysbiosis by: acquiring a sample of microbiota existing in a patient's GI tract, creating a profile of existing microbiota corresponding to levels of selected microbial phyla and/or proportions of selected microbial phyla, wherein the microbial phyla are selected from phyla Firmicutes, Bacteroidetes, Actinobacteria, Proteobacteria, Fusobacteria, and Verrucomicrobia, and administering a therapeutic microbiota to the patient that includes levels and/or proportions selected to urge proliferation of existing microbiota toward a normal microbiota profile.

### BRIEF DESCRIPTION OF THE DRAWINGS

FIG. 1 is a sectional illustration of a portion of a wall of an individual's duodenum, showing the mucous layers of the surface of the lumen that carries the residing microbiota, the mucosa, submucosa, villi, and other anatomical layers of the intestinal wall.

FIG. 2 is an enlarged illustration of mucous layers, microbiota, mucosa, and submucosa of FIG. 1.

FIG. 3 is an illustration of a portion of an individual's gastrointestinal tract showing a method of using a gastroscope and ablation catheter to position the working end of the catheter in the duodenum of the individual.

FIG. 4 is an illustration of the handle of the gastroscope and the ablation catheter of FIG. 3.

FIG. 5 is an enlarged view of the working end of the catheter and spaced apart occlusion balloons.

FIG. 6 is a sectional view of a catheter shaft taken along line 6-6 of FIG. 5, showing the flow channels therein.

FIG. 7A is an enlarged view of the duodenum of FIG. 3 showing the working end of the catheter and the occlusion balloons expanded in the individual's duodenum.

FIG. 7B is a view of the duodenum as in FIG. 7A showing the working end of another variation of a catheter with a scalloped occlusion balloon expanded in the individual's duodenum.

FIG. 8 is an illustration of a portion of an individual's gastrointestinal tract showing a method of implanting an implantable device of the invention adapted to deliver restorative, therapeutic microbiota to the duodenum of the individual.

FIG. 9 is an illustration of the individual's gastrointestinal tract as in FIG. 8, showing a variation of an implantable device that carries a communication component for wireless communication with a smartphone or other device to operate a micropump in the implant or other mechanism for modulating the release of the restorative microbiota to the duodenum of the individual.

FIG. 10 is an illustration of an individual's colon showing a variation of a treatment device for performing a lavage to remove and eliminate the mucous and residing microbiota of a targeted region of the colon.

### DETAILED DESCRIPTION OF THE INVENTION

The present invention relates to systems and methods for the treatment or prevention of dysbiosis in the gastrointestinal (GI) tract of an individual. Such GI tract dysbiosis can be defined as a physiological state in which the microbiota profile of at least a portion of an individual's GI tract is not in a normal state or that the profile differs significantly from a corresponding GI tract microbiota profile that is typical of a normal, healthy individual. Such a profile of an individual's GI tract microbiota can be represented by quantities of various taxonomic groups and, ultimately, species, and also can be evaluated the relative levels of various groups of microbiota, as described further below.

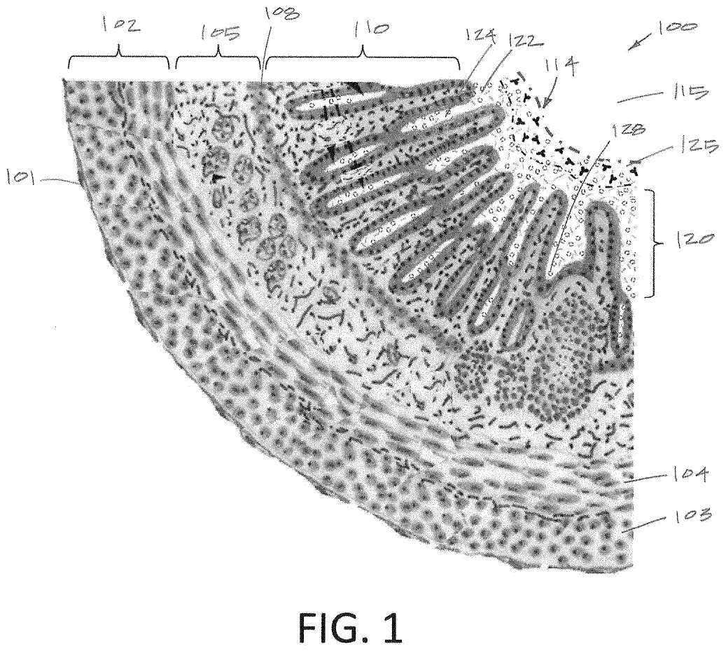

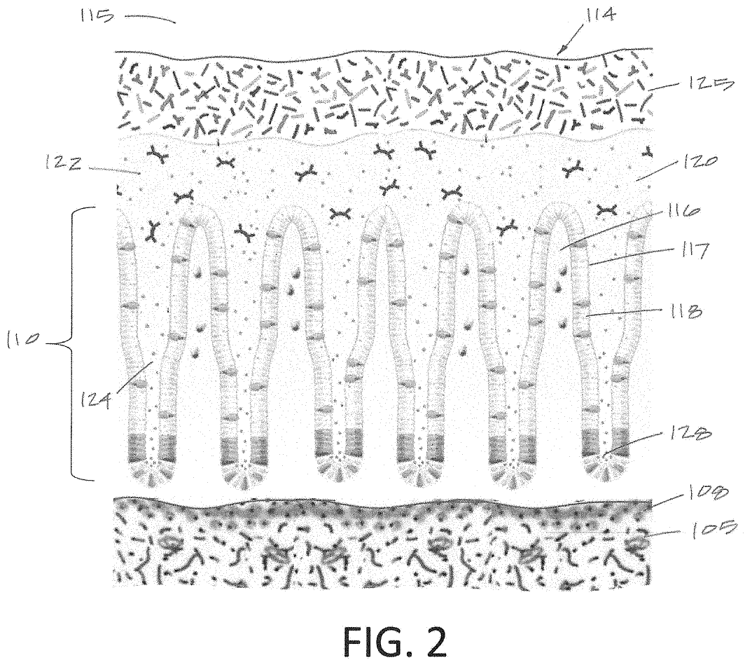

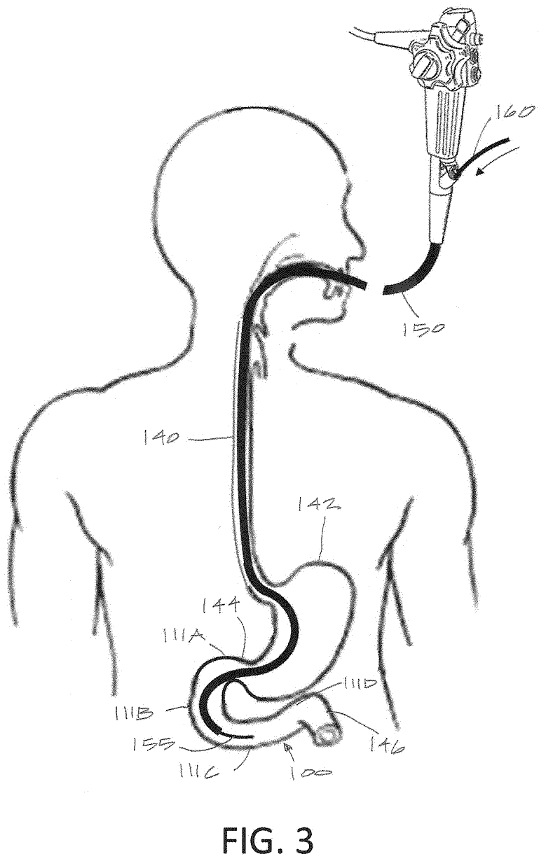

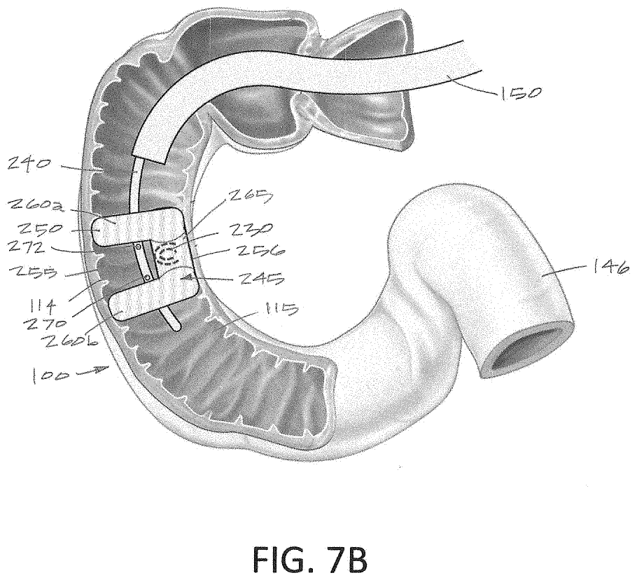

FIGS. 1 and 2 are schematic sectional illustrations of a portion of small intestine and, more particularly, of an individual's duodenum 100. In FIG. 1, the wall of the duodenum 100 includes the outer tunica serosa 101, the mucularis 102 comprising an outer longitudinal layer 103 and an inner circular layer 104, the submucosa 105, the muscularis mucosae 108, and the mucosa 110. The duodenum, as shown in FIGS. 3 and 7A has four sections with borders being delineated by angular course changes, commonly called the first part 111A or superior part, the second part 111A or descending part, the third part 111C or horizontal part, and the fourth part 111D or the ascending part.

The mucosal lining or mucosa 110 of the small intestine is well adapted for the function of nutrient absorption by anatomical structures that increase the surface area for trans-mucosal absorption at three levels. The inner surface of duodenum has plicae circulares or circular folds 112 (see FIG. 7A) that increase the surface area by approximately 3-fold. The mucosa 110 projects from the circular folds 112 of the luminal surface 114 into the lumen 115 with villi 116 that, are finger-like structures approximately 1 mm in length (FIGS. 1, 2, and 7A). The villi 116 increase the surface area by an additional 10-fold. Finally, the surface of each villus is covered with epithelium 117 with epithelial cells (FIG. 2) known as enterocytes where absorption takes place across the enterocyte barrier. Small hair-like filaments known as microvilli 118 project from the luminal surface villi into the lumen 115 (FIG. 2). The microvilli 118 increase the surface area for absorption by an additional 20-fold. These three anatomical structures combine to increase the surface area of the small intestine by approximately 600-fold.

FIGS. 1 and 2 further illustrate the luminal surface 114 of lumen 115, and contents of the luminal surface which includes a mucous layer 120 consisting with an outer mucous portion 122 and an inner mucous portion 124. The residing microbiota 125 is on the luminal surface or partly in the mucous layer 120. The inner mucous portion 124 descends around the villi 116 toward the crypts 128 at the base of the villi 116.

An objective of the invention is to ablate the entirety of the residing microbiota 125 and at least a portion of the mucous layer 120 without ablation of the submucosa 105. The ablation of the microbiota 125 and the mucous layer 120 can also ablate the villi 116 and endothelium that is immersed in the mucous layer 120. Another objective of the invention is to administer to the patient a restorative or replacement microbiota which will re-populate the ablated region as a new mucosa regenerates over a period of 1 to 4 weeks in the methods described below.

In a variation, the introduction of the restorative microbiota can be accomplished over a time interval of 1 week to 180 days to return the patient to a more normal GI tract microbiota profile as described below. The divergence of a patient's GI microbiota profile away from a normal profile has been observed in many disorders, diseases, and pathological conditions. Thus, treating GI tract dysbiosis corresponding to the use of systems and methods of the invention can treat medical conditions selected from the group consisting of: gastrointestinal inflammation, metabolic syndrome, obesity, prediabetes, Type 2 diabetes, Type 1 diabetes, insulin resistance, non-alcoholic fatty liver disease, non-alcoholic steatohepatitis, obesity and related disorders, gastroesophageal reflux disease, Barretts esophagus, irritable bowel syndrome, Crohn's disease, ulcerative colitis, celiac disease, constipation, diarrhea, colorectal cancer, polycystic ovarian syndrome, coronary artery disease, heart disease, stroke, cognitive decline, dementia, Alzheimer's Disease, fertility issues, menstrual dysfunction, cancers, eczema, sleep apnea, multiple sclerosis, arthritis, rheumatoid arthritis, asthma, chronic fatigue syndrome; autism, atopic dermatitis, psychiatric disorders (e.g., depression and anxiety) and combinations thereof.

Now turning to FIGS. 3 to 7A, a system and method of the invention for ablating the contents of the luminal surface 114, the residing microbiota 125 and the mucous layer in a patient's duodenum 100 is illustrated. FIG. 3 is a schematic illustration that shows a patient's esophagus 140, stomach 142, pylorus 144, duodenum 100, and jejunum 146 with a gastroscope or endoscope 150 being introduced into the patient's duodenum 100. Further, an elongate catheter shaft 155 of a vapor delivery catheter 160 is introduced through a working channel 162 in the endoscope 150.

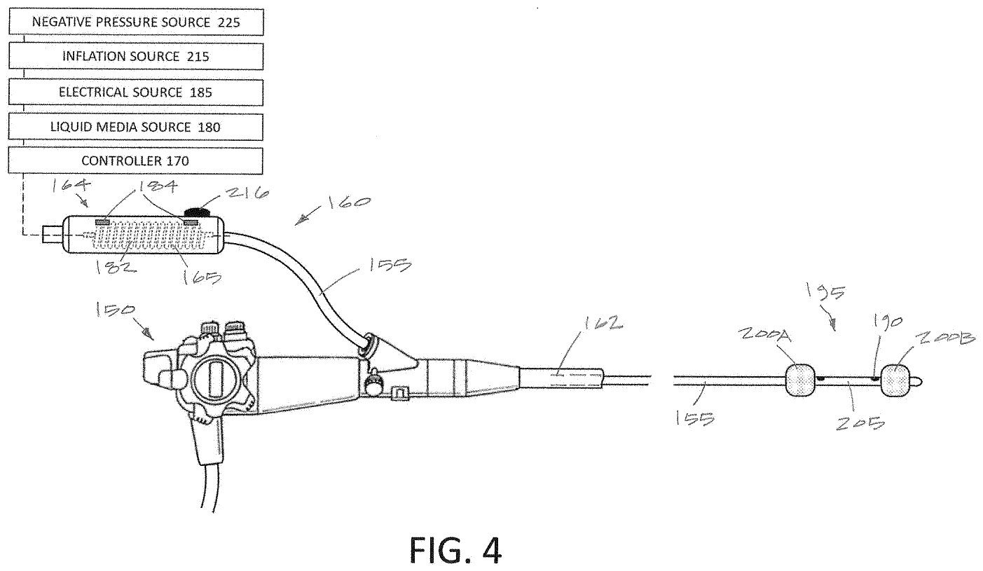

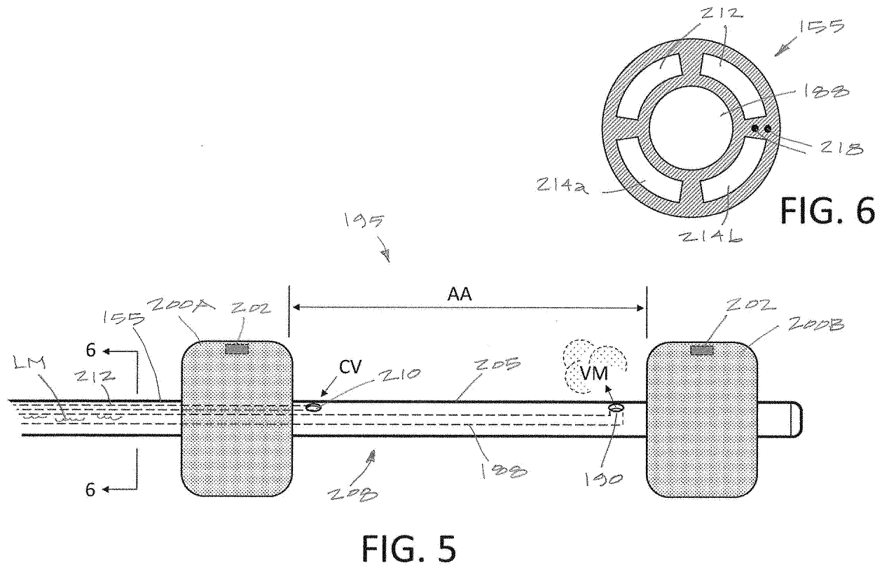

The catheter 160 as shown in FIGS. 4 and 5 is configured to deliver a condensable, thermal vapor media, such as sterile, condensable water vapor, to a targeted region of the gastrointestinal tract. Variations of probes and catheters of this type are described in commonly-owned U.S. Pat. Nos. 11,284,932; 11,284,931; 11,179,187; 11,141,210; 11,129,664; 10,548,653; 10,499,973; 9,943,353; and 8,579,892 and commonly-owned and co-pending U.S. Patent Applications Ser. Nos. 17/304,102; 17/457,501; 17/647,835 and 63/367, 293, all of which are incorporated herein by this reference.

In a variation as shown in FIG. 4, the catheter handle 164 carries a heating mechanism 165, which is capable of instantaneously converting an inflow of sterile flowable liquid media LM, such as sterile water, into a flowable thermal vapor media VM, such as water vapor (FIG. 4). More, in particular, a controller 170 is provided that when actuated causes a liquid media source 180 including a syringe pump to pump the sterile liquid media LM into the heating mechanism 165 wherein the liquid media is instantly converted to thermal vapor media, such as water vapor. The controller 170 operates the syringe pump to provide a very precise flow rate of the liquid media into the heating mechanism 165. In a variation, the heating mechanism 165 comprises a helical tubing element 182 into which the liquid media is pumped. The material of the helical tubing element 182 is fabricated from an electrically resistive material, such as stainless steel, Inconel, or the like, that, when heated, will instantly convert the flow of sterile liquid media into water vapor. The helical tubing element 182 is suitable since it is axially compact and can be carried within the catheter handle 164 with electrical leads from an electrical source 185 coupled to opposing ends of the helical tubing element 182.

The helical tubing element 182 has one or more temperature sensors 184 attached thereto that send temperature signals to the controller 170 to control or modulate either or both (i) electrical current delivered from the electrical source 185 to the heating mechanism 165 and (ii) the flow rate of liquid media into the heating mechanism 165 to thereby control the outflow of heated vapor media from the heating mechanism which then flows through a vapor inflow channel 188 to an outlet 190 in the working end 195 of the catheter shaft 155 (FIGS. 4 to 6). In a variation, a temperature sensor 184 is coupled to both the proximal and distal ends of the helical tubing 182 to monitor the temperature of the helical tubing with the fluid flowing therethrough. In another variation, another temperature sensor, 184 is coupled to the middle portion of the helical tubing 182, where the controller 170 is configured with algorithms that monitor signals from one to three temperature sensors. In response to the temperature signals, the controller 170 can modulate energy delivery to the helical tubing 182 to maintain the optimal temperature to instantly cause the liquid-to-vapor phase change. In a typical variation, the controller 170 and heating mechanism 165 can provide an outflow of vapor media VM from the vapor outlet 190, having a vapor quality that is at least 80% water vapor, at least 85% water vapor, or at least 90% water vapor with less than a 10% variation in the vapor quality over a selected time interval of at least 5 seconds.

As can be seen in FIGS. 4, 5, and 6, the working end 195 of the catheter shaft 155 carries proximal and distal occlusion balloons, 200A and 200B, that are configured for expansion in a gastrointestinal lumen 115. The occlusion balloons 200A, 200B can be compliant or non-compliant and are configured to occlude a lumen 115 than can range from 18 mm to 24 mm in diameter. When the occlusion balloons are fabricated of a non-compliant material, a series of catheters can be manufactured with occlusion balloons that have selected different diameters, for example, a first catheter with 18-20 mm diameter occlusion balloons, a second catheter with 20-22 mm balloons, a third catheter with 22-24 mm balloons, etc. In the case of compliant occlusion balloons, it is possible that each occlusion balloon 200A, 200B can be expanded to a selected diameter within the range of 18 mm to 24 mm so that a single manufactured catheter 160 is suitable. Either type of occlusion balloon can be configured with a sensor 202 in the surface of either or both balloons to sense engagement of the balloon surface with the wall of the lumen 115 (FIG. 5). Such a sensor 202 can comprise a capacitance sensor, an impedance sensor, another form of electrical contact sensor or a light energy contact sensor.

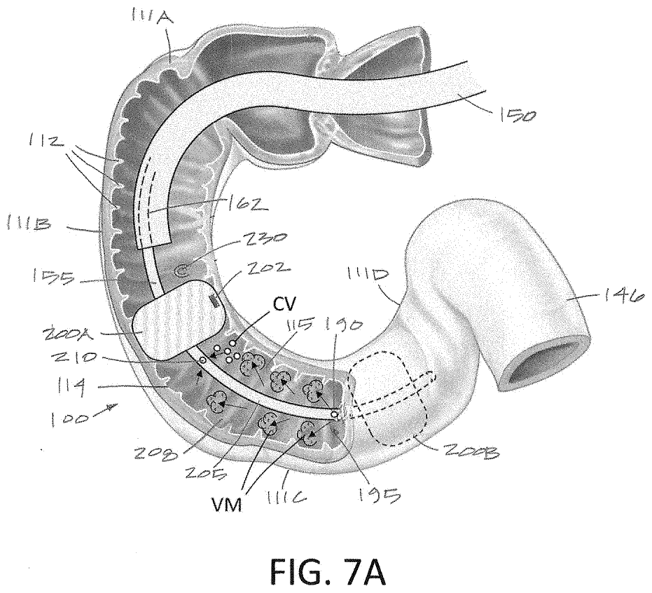

Referring to FIGS. 5 and 6, the axial length AA of the inter-balloon catheter shaft portion 205 between the occlusion balloons 200A, 200B can range from 2 cm to 25 cm and is typically between 5 cm to 10 cm. The vapor inflow channel 188 in the catheter shaft 155 has at least one vapor outlet 190 in the inter-balloon catheter shaft portion 205 that introduces the condensable vapor media VM into the luminal space 208 (FIG. 7A) between the occlusion balloons 200A, 200B, wherein the targeted region of the GI tract comprises the luminal surface 114 of the lumen 115 between the expanded occlusion balloons. The luminal surface 114 of lumen 115 comprises the mucous layer 120 and microbiota 125 as shown in FIGS. 1 and 2. Often, there is a single vapor outlet 190 disposed in the inter-balloon catheter shaft portion 205, as shown in FIGS. 5 and 6, but a plurality of vapor outlets may be provided axially and/or circumferentially around the shaft portion 205. Additionally, at least one outflow port 210 is provided in the inter-balloon catheter shaft portion 205 that is adapted to extract condensate or water droplets (i.e., the condensed vapor) from the inter-balloon space 208 through outflow channel 212 in the catheter shaft 155 (FIGS. 5 and 6). In a variation shown in FIG. 5, a vapor inflow outlet 190 is positioned in a first end of the intra-balloon catheter shaft portion 205 and a condensate outflow port 210 is in the opposing, second end of the inter-balloon catheter shaft 205 to create a circulating flow. Thus, the vapor inflows and the condensate outflows provide a form of circulating flow that can reduce the time interval needed for ablation of the targeted luminal surface 114, mucosa 120 and microbiota 125 (FIGS. 1 and 2). The circulating flow can function to extract a large portion of the condensate which in turn decreases the required energy delivery to the luminal surface 114 of lumen 115 to ablate to a selected depth because if the condensate were not extracted, the condensate (or liquid water) would otherwise need to be heated together with the targeted contents of the luminal surface 114 including the microbiota 125 of the lumen 115.

FIG. 6 is a sectional view of the catheter shaft 155 showing a variation of an extrusion that carries the vapor inflow channel 188, the condensate outflow channel 212 comprising dual passageways, and an independent inflation channel 214a, 214b for the respective proximal and distal occlusion balloons 200A and 200B. In FIG. 6, it can be seen that the vapor inflow channel 188 is disposed of in the center of the catheter shaft 155, with the other surrounding channels 212, 214a, and 214b having an arcuate shape around the central channel 188. The surrounding channels 212, 214a, and 214b are filled with air or condensate during use which provides a thermally insulating layer around the central vapor inflow channel 188, which is advantageous in helping maintain the quality of the vapor media VM flowing in the central inflow channel 188. In another variation, the catheter shaft 155 can carry a single inflation channel (not shown) for inflating both occlusion balloons 200A, 220B contemporaneously rather than independently, wherein the catheter shaft 155 would then be configured with two or three arcuate channels instead of four arcuate channels as shown in FIG. 6. The occlusion balloons are inflated by an inflation source 215 that be controlled by the controller 170 or can Be manually inflated by a syringe. Electrical leads 218 are carried in the shaft 155 the extend to the contact sensors 202.

Referring to FIGS. 3 and 7A, a method of the invention includes introducing the working end 195 of a vapor delivery catheter 160 catheter into the duodenum 145 to a targeted region of the lumen 115 thereof. The physician then, under endoscopic vision, expands the occlusion balloons 200A, 200B, which can be done sequentially or contemporaneously. In a variation, the physician positions the distal occlusion balloon 200B in a desired location under endoscopic vision and then observes the inflation of this occlusion balloon 200B within the lumen 115 of the duodenum 100. Subsequently, the physician moves the endoscope 150 proximally and then observes the inflation of the proximal occlusion balloon 200A.

Referring to FIG. 7A, after the occlusion balloons 200A, 200B are in position and expanded, the physician actuates the controller 170, for example, by means of an actuator button 216 on the catheter handle 164 (FIG. 4). A pre-set on the controller 170 is selected to deliver the thermal vapor media VM for a time interval ranging between 2 seconds and 20 seconds. The controller 170 also has pre-sets on its user interface to deliver a selected number of cal/sec, which is controlled by the flow rate of the sterile liquid media LM into and through the heating mechanism 165. For any selected flow rate, controller 170 can control the electrical source 185 to apply the suitable electrical energy to the resistive heating mechanism 165 to convert the sterile liquid media inflow into high quality thermal vapor media, such as water vapor. As described above, at least one temperature sensor 184 coupled to the heating mechanism 165 sends temperature signals to the controller 170, which then modulates the resistive heating of the heating mechanism 165 responsive to the temperature signals.

Referring to FIG. 7A, the inflow pressure from the liquid media source 180 and its syringe pump then further pumps the thermal vapor media VM through the inflow channel 188 in the catheter shaft 155 to the outlet 190 in the inter-balloon catheter shaft portion 205. The thermal vapor then condenses in the lumen 115 to apply energy to the targeted luminal surface 114 of the lumen 115, wherein the vapor-to-liquid phase change releases large amounts of energy and uniformly ablates a thin layer of the luminal surface 114 of the lumen 115, which includes the residing microbiota 125 and the mucous layer (FIGS. 1-2). The condensate or condensed vapor CV is at least in part removed from the inter-balloon space 208 in the lumen 115 through the outflow port 210 and outflow channel 212 in the catheter shaft 155, as described above. The outflow port 210 and output channel 212 can be a passive channel where any pressure in the inter-balloon space 208 causes an outward flow of the condensate. In another variation, a negative pressure source 225 can communicate with the outflow channel 212 and be controlled by the controller 170 to extract condensate from the inter-balloon treatment space 208 (FIGS. 4, 5, and 7A). In a typical treatment, the delivery of vapor media VM to ablate the contents of the luminal surface 114 and microbiota 125 would be from 2 to 20 seconds of vapor delivery and often from 5 to 10 seconds.

As can be seen in FIG. 7A, the occlusion balloons 200A, 200 engage the wall of the GI tract lumen 115 so that the targeted region of the luminal surface 114 between the occlusion balloons remains in its natural state with the circular folds 112 undisturbed. This is very advantageous as when the condensable vapor media VM is introduced through the outlet 190 in the inter-balloon catheter shaft portion 205, the vapor-to-liquid phase change causes the release of energy, and ablation, at all exposed luminal surface 114 of the lumen 115, where the condensation occurs. Thus, the vapor's phase change will ablate the contents of the luminal surface 114 comprising the microbiota, mucous layer 120 and mucosa 110 uniformly in and around the circular folds 112 in their natural state. The ability of vapor to ablate all luminal surfaces of the lumen 115 uniformly differs significantly from other potential forms of energy delivery that might be considered. For example, other means of delivering thermal energy or cryogenic freezing can consist of a hot water balloon, cryo-balloon or a balloon with a surface that is heated by RF electrodes, resistive heating elements, microwave energy, light energy, or the like, which could be expanded to engage the surface of the lumen 115 (FIG. 7A). However, any expandable member such as a balloon would compress the circular folds 112 and villi 116 in the lumen 115 and prevent ablation to a uniform depth in the surface layers of the lumen 115.

In another important aspect of a method of the invention, an entire selected length of the duodenum 100 and optionally a region of a jejunum 146 can be continuously and uniformly ablated by sequentially repositioning the working end 195 of the catheter 160. In a variation, the physician typically would position the catheter working end 195 and occlusion balloons 200A, 200B in a distal location in the duodenum 100 or jejunum 146. For an initial ablation, as described above, physician would observe the expansion of the distal balloon 200B, then optionally relocate the endoscope and observe the expansion of the proximal balloon 200A and then introduce the vapor media VM to complete the first ablation. Thereafter, the physician would collapse the occlusion balloons 200A, 200B and move that working end 195 in the proximal direction. The physician would then endoscopically observe the positioning of distal balloon 200B in the lumen 115 so that the second ablation would slightly overlap the first ablation. The physician would then expand the proximal occlusion balloon 200A and introduce vapor media VM to complete the second ablation. The physician would repeat this re-positioning of the working end 195 to provide a continuous ablation of the luminal surface 114 of the lumen 115. In this sequential ablation method, in one of the re-positioning steps, either the proximal balloon 200A or the distal balloon 200B is positioned to cover the ampulla of Vater 230 so that the region around the ampulla would be protected from the ablation (FIG. 7A). By this method, the physician can ablate luminal surface 114, mucosa 120 and microbiota 125 continuously over a length of the small intestine that is at least 20 cm, at least 25 cm or at least 30 cm. It is believed to be important to ablate the residing microbiota 125 uniformly over such an extended length of the GI tract so that when restorative microbiota 425 is introduced as described below, there will be little or no residing microbiota 125 to compete with the progressive growth of the restorative microbiota 425.

As can be understood, the method of sequential ablation allows for ablation of the residing microbiota 125 and mucous layer 120 in the first part 111A, second part 111B, third part 111C, and fourth part 111D of the duodenum 100 (FIG. 7A). It should be appreciated that the sequential ablation can progress from a proximal position to a distal position which is the reverse of the above description. The number of sequential ablations can range from 2 to 8 depending on the length of treatment space 208 between the occlusion balloons 200A, 200B, which can be from 5 cm to 25 cm, as described above.

In a variation, the catheter shaft has a diameter of 2.8 mm or less so that it can be introduced through the working channel of a conventional, commercially available gastroscope 150 of the type shown in FIGS. 3 and 4. In a variation, the conventional gastroscope has a working channel length of approximately 100 centimeters. Thus, the length of the catheter shaft 155 extending from handle 164 is 100 cm from the proximal balloon, and often at least 110 cm, at least 120 cm, or at least 130 cm so that the working end 195 of the catheter 160 can be extended distally from the distal end of the gastroscope 150 a sufficient distance to observe the expansion of the occlusion balloons 200A, 200B.

In general, a catheter 160 of the invention for treating GI tract dysbiosis comprises a handle, a flexible catheter shaft extending from the handle having a proximal end and a working end carrying at least a proximal occlusion balloon and a distal occlusion balloon wherein the proximal occlusion balloon is spaced apart at least 100 cm from said proximal end of the catheter shaft, and a heating mechanism capable of converting a flow of liquid media to a flow of vapor media that exits the catheter shaft at an outlet located between the proximal and distal occlusion balloons wherein the vapor media has a vapor quality of at least 80% vapor, 85% vapor or 90% vapor and wherein the flow of vapor media from the outlet delivers at least 10 cal/sec to a GI tract lumen in which the proximal and distal occlusion balloons are expanded. In variations, the catheter 160 is capable of providing a flow of vapor media from the outlet that delivers at least 5 cal/sec, 25 cal/sec, or 50 cal/sec.

In another variation, the invention comprises a custom endoscope device that has a longer shaft than a conventional gastroscope 150, with the custom endoscope having an increased shaft length. Often, a conventional gastroscope shaft is not long enough to reach the third part 111C or fourth part 111D of the duodenum 100 or the jejunum 146. In a variation where it is deemed important to have in endoscopic viewing in the distal part of the duodenum 100 or in the jejunum 146, an endoscope of the invention has a shaft with a length of at least 120 cm, at least 130 cm, or at least 140 cm. In a variation, such an endoscope device can comprise an integrated catheter that carries one or more image sensors and with a component or section of the working end carrying the occlusion balloons as disclosed in commonly-owned U.S. Patent Applications 63/367,293; Ser. Nos. 17/647,835; 17/457,501 and 17/304,102. In another variation, a single-use endoscope with a suitable length as described above can be provided with a 2.5 to 5 millimeter working channel for receiving the catheter, the type shown in FIGS. 4 to 7A with an extended length to be used with the endoscope.

Referring to FIG. 7B, another variation catheter 240 with working end 245 that includes a scalloped occlusion balloon structure 250 that is configured to allow vapor ablation of the luminal surface 114 on a first side 255 of a gastrointestinal lumen 115 while preventing ablation of a second, opposing side 256 of the lumen 115. In a variation, the occlusion balloon 250 comprises a proximate balloon portion 260a and distal balloon portion 260b that are configured to expand to engage the wall of the lumen as shown in FIG. 7B. As can be seen in FIG. 7B, the occlusion balloon 250 has a medial portion comprising an axially extending element 265 between the proximal balloon portion 260a and the distal balloon portion 260b. The axially extending element 265 can be inflatable or non-inflatable and is adapted to cover a side 256 of the lumen 115 when the proximal and distal balloon portions 260a, 260b are expanded. The scalloped occlusion balloon 250 this is useful for protecting in ampulla of Vater 230 that was described above. A vapor inflow channel 270 and condensate outflow channel 272 are provided to function as described above. This type of occlusion balloon structure 250 of FIG. 7B can be carried on an independent catheter 240 as shown in FIG. 7A or can be carried on a type of catheter 160 as shown in FIG. 7A that carries first and second occlusion balloons 200A, 200B as described previously. The scalloped occlusion balloon structure 250 can be positioned proximally or distally from the paired occlusion balloons 200A, 200B or ca replace either of the balloons 200A, 200B. In any event, such an occlusion balloon structure can be used in a situation where the objective is ablate the luminal surface 114 and residing microbiome 125 over a continuous length of a duodenum 100 only limited by a region around the ampulla of Vater 230 which is protected from ablation.

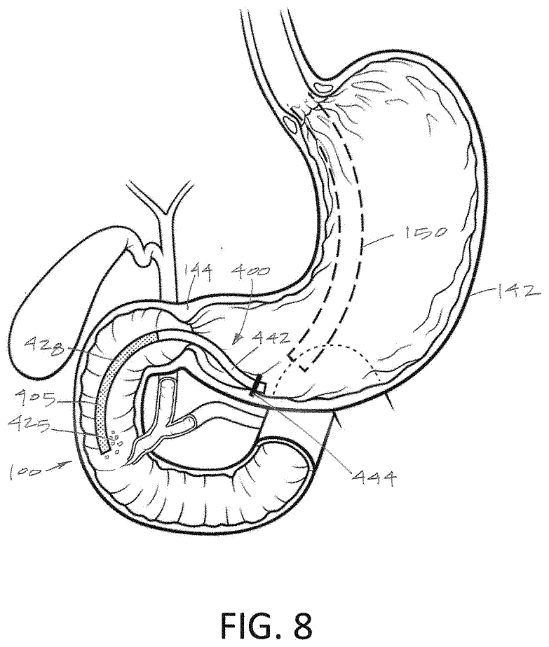

In another aspect of the invention, FIG. 8 illustrates an implant device 400 that is implanted in the patient and adapted to deliver restorative microbiota 425 to the GI tract over a selected time interval. In FIG. 8, it can be seen that an endoscope 150 in phantom view has been introduced in a trans-esophageal approach. The implant device 400 can be delivered through a working channel in the conventional endoscope 150, or a clip applier tool can be introduced through the working channel with the implant device 400 delivered outside of the endoscope. It should be appreciated that a nasogastric approach is also possible with a smaller diameter endoscope. In FIG. 8, the implant device 400 is shown as an elongated member that carries at least one reservoir 405 therein which carries the restorative microbiota 425 that is described further below. The microbiota 425 can be released over a time interval through an agent-release portion 428 of the implant device 400 which can consist of micropores or biodegradable surfaces overlying reservoirs carrying the restorative microbiota 425. In a variation, the implant device 400 has proximal end 442 that is adapted for fixation to the wall of the stomach 142 with a clip 444 as is known in the art. A clip applier tool (not shown) is introduced through the working channel 162 of the endoscope 150 to affix the clip 444. As indicated in FIG. 8, a proximal portion 442 of the implantable device 400 that is disposed in the stomach 142 may not carry micropores or reservoirs with biodegradable surfaces for releasing the microbiota 425 as the release of microbiota in the stomach may be degraded. In a variation, a distal portion 428 of the implant device 400 is positioned in the patient's duodenum 100, which is the preferred site for releasing the restorative microbiota 425. In another variation, the implant device 400 can be tethered in the duodenum 100.

In a variation, the restorative microbiota 425 comprises an effective amount of bacteria from any gram positive family or from any gram negative family. The effective amount of bacteria can be selected from two or more different taxonomic families selected from the group of Bacteroidaceae, Acintobacter, Firmacutes, Protetobacter, Lactobacillus, Fusibacter, Ruminococcaceae, Rikenellaceae. Clostridiaceae and Lachnospiraceae. Typically, such bacteria comprise a processed fecal composition wherein such processing is known in the art. On a species level, the restorative microbiota 425 can include an effective amount of Anaerobutyricum Soehngenii, an anaerobic Gram-positive, catalase-negative bacterium as described in Gilijamse, P. W., Hartstra, A., Levin, E. et al. “Treatment with Anaerobutyricum soehngenii: a pilot study of safety and dose-response effects on glucose metabolism in human subjects with metabolic syndrome”; npj Biofilms Microbiomes 6, 16 (2020), and (https://doi.org/10.1038/s41522-020-0127-0) and Koopen A, Witjes J, Wortelboer K, et al. titled “Duodenal Anaerobutyricum soehngenii infusion stimulates GLP-1 production.” Gut Epub Oct. 25, 2021, which articles are incorporated herein by this reference. In a variation, the restorative microbiota 425 includes a processed human fecal composition. Typically, the restorative microbiota 425 comprises a lyophilized composition that is processed and fabricated as is known in the art. Research companies such as Creative BioLabs Inc, at 17 Ramsey Road Ste. 202, Shirley, N.Y. 11967 can be engaged to produce some or all types of restorative microbiota 425. Other companies that can provide tools for profiling the residing microbiota and/or providing restorative microbiota are Diversigen Inc., 600 County Road D West, Suite 8, New Brighton, Minn. 55112; Rebiotix Inc., 2660 Patton Road. Roseville, Minn. 55113; DNA Genotek, 3000-500 Palladium Drive, Ottawa, Ontario, Canada K2V1C2; and Compound Solutions, Inc., 1930 Palomar Point Way, Suite 105, Carlsbad, Calif. 92008. The NIH Human Microbiome Project (https://hmpdacc.org/) has characterized the microbiota from healthy individuals across the gastrointestinal tract for comparison and also has data relating to the GI tract microbial enrichments, depletions, and dysbioses in microbial metabolic activities.

In the variation shown in FIG. 8, it should be appreciated that the implanted device 400 is adapted for temporary implantation, wherein the clip 444 is intended to maintain the implant 400 in position for a period of time ranging from one week to six months. The clip 444 is designed to eventually slough off the stomach lining and, together with the remainder, if any, of the biodegrading implant 400 and pass through the patient's gastrointestinal tract.

In a variation, the implant device 400 may be substantially short in length and be adapted to release the microbiota into the first part 111A of the duodenum 100, or the device can have length ranging from 10 cm to 50 cm to extend through the entire length of the duodenum 100 and the jejunum 146.

In another variation, the implant device 100 can be adapted for temporary fixation with a clip or other fastener to a wall of the patient's pylorus 144 or a wall of the duodenum 100. In another variation (not shown), the implant device may carry a larger reservoir and be configured with a proximal implant portion that resides in the patient's stomach 142. In such a variation, the implant may carry a donut-shaped element for positioning proximal to the pylorus 144. Such an implant variation would typically be adapted for introduction and retrieval through a trans-esophageal approach using an endoscope. In a variation, such a semi-permanent implant can include a refillable reservoir for carrying restorative microbiota 425.

In a variation, the implant device 400 can consist of an elongated sleeve, tubular member, ribbon, or filament of a flexible polymeric material, as shown in FIG. 8, with an interior chamber or reservoir carrying the restorative microbiota 425. At least a portion of the surface of the polymeric material overlying the interior chamber is configured with micropores that allow for the release of the microbiota therethrough. The implant may also comprise a plurality of elongated members tethered by a clip 444.

In another variation, an implant device configured as in FIG. 8 can comprise a biodegradable member with a plurality of chambers therein that carry restorative microbiota 425. In this variation and other variations above, the polymer surfaces overlying the restorative microbiota 425 can be configured to biodegrade or bio-erode at different rates to allow for the release of the microbiota 425 over an extended time interval. Such bioabsorbable, biodegradable, or bio-erodible polymers have been widely used medical devices or for the delivery of drugs. Suitable the polymers are Poly-L-lactic acid (PLLA), polyglycolic acid (PGA), poly (D, L-lactide/glycolide) copolymer (PDLA), and polycaprolactone (PCL). Various degradation rates for such polymers can be designed.

In a variation, the implant device 400 can be designed to release the restorative microbiota 425 over a selected time interval, where the implant 400 can commence the release within 1 hour of the time of implanting the device, and the selected time interval for ending the release of said microbiota can be between 1 day and 180 days from the time of implanting the device 400.

In general, a method corresponding to the invention for treating GI tract dysbiosis comprises ablating the residing microbiota of a targeted portion of a GI tract of a patient with a GI tract dysbiosis and administering a restorative microbiota to the patient comprising an effective amount of bacteria from two or more different taxonomic families selected from the group of Bacteroidaceae, Acintobacter, Firmacutes, Protetobacter, Lactobacillus, Fusibacter, Ruminococcaceae, Rikenellaceae. Clostridiaceae and Lachnospiraceae. The method can include administering the restorative microbiota comprising Anaerobutyricum Soehngenii. The effective amount or dose of restorative microbiota is the amount necessary to re-colonize or re-populate the targeted region of the GI tract, which will be in competition with the residing microbiota in portions of the GI tract adjacent to the ablated region that will also be migrating and attempting to re-colonize the targeted region. For this reason, the timed release and continuous release of restorative microbiota 425 over a time interval may be optimal, or a continuous release from the daily ingestion of capsules may be adequate.

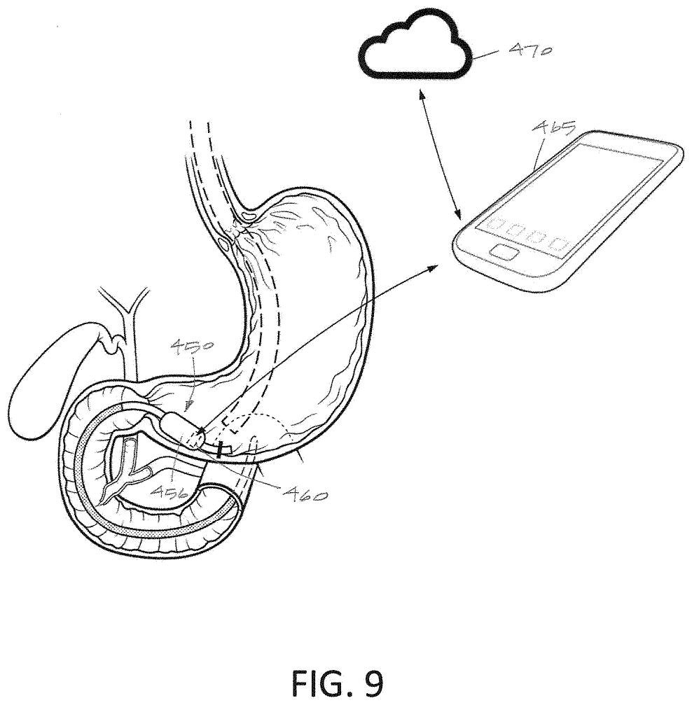

Now turning to FIG. 9, another variation of implant device 450 is shown where a proximal region 456 of the implant carries a communication component 460 for wireless communication with a remote device 465 such as a smartphone or tablet. This variation includes a micropump that is adapted to pump restorative microbiota 425 from a reservoir in the device to outlets therein in response to signals from the remote device 465. Such a remote device 465 can also communicate with the cloud 470 which can provide access to remote physicians to allow adjustment or modulation of dosimetry or agent release in response to questionnaires or responses from the individuals concerning his or her state of health.

Another aspect of the invention includes profiling the residing GI tract microbiota 125

(FIGS. 1-2) prior to ablation thereof or the administration of restorative microbiota 425. Such a profile of a residing microbiota may involve any convenient means by which the levels of microorganisms or groups of microorganisms in the GI tract of the patient can be identified and/or quantified. Such a microbiota profile would be prepared with in vitro methods using a sample taken from the GI tract. In a variation relevant to this disclosure, the sample would be acquired by a swab, rinse, aspirate, scrape or biopsy of a small intestine surface. Other treatments using the methods of the invention in other regions of the GI tract would acquire microbiota samples in the targeted region. For example, the human GI tract can be subdivided into the upper GI tract consisting of the esophagus and stomach, and the lower GI tract or the intestinal tract consisting of the duodenum, jejunum, ileum, cecum, colon, and rectum. Treatments of the esophagus and colon are possible using the methods of the invention, in which case profiles would be prepared for the targeted region from samples acquired from that region. For example, microbiota and mucous would be collected from the esophagus for evaluating the treatment of esophageal disorders. For potential treatments of the colon, fecal samples would be collected for profiling the residing microbiota therein.

In a method of the invention, in the event a profile of the residing microbiota 125 as described above shows that the profile is not highly differentiated from a normal profile, a method of the invention is to provide the timed release of restorative microbiota 425 from an implant device of FIGS. 8-9 without the ablation of the residing microbiota 125. In such a case where the profile of residing microbiota is not highly differentiated from normal, a method of the invention can include administering the restorative microbiota 425 by means of liquid compositions taken orally or introduced through an endoscope, capsules, aerosols, enemas, suppositoriesor the like rather than using an implant device 400, 450 as shown in FIGS. 8-9.

FIG. 4 illustrates the heating mechanism 165 carried in the handle 164 of the catheter 160. It should be appreciated that a heating mechanism comprising helical or spaced apart, resistively heated elements can also be carried in a medial portion of the catheter shaft 155 or in a distal region of the catheter shaft 155. In another variation, a primary resistive heating mechanism 165 can be carried in the catheter handle 164, and a supplementary heating mechanism can be carried in a distal region of the catheter shaft to optimize vapor quality (cf. FIG. 4). A heating mechanism 165 carried in a medial or distal region of the catheter shaft 155 can consist of a flexible helical element exposed to the vapor inflow channel 188 or can consist of a series of ring-like resistively heated elements with exposed surfaces in the vapor inflow channel 188 which again allows for flexibility of the catheter shaft 155. Any of these variations with one or more heating mechanisms can provide high-quality vapor as described above.

The above methods of ablating the luminal surface 114, mucous 120 and residing microbiota 125 of a patient's GI tract describe the advantages of using the condensable vapor CV (FIG. 7A), where the vapor-to-liquid phase change applies an enormous amount of energy uniformly over a luminal surface. It can be understood that sterile liquid vapor is an optimal liquid media LM to vaporize, but other fluids can be used or added to liquid water, such as alcohol or other chemical compositions, to provide the ablative effect. Also, it should be appreciated that the scope of the invention includes using alternative forms of energy delivery, both thermal and cryogenic, such as heated or cooled balloons that engage the luminal surface 114, as well as energy applicators that do not engage the luminal surface, all of which are followed by administration of restorative microbiota 425. Such other forms of energy delivery can include RF devices, microwave devices, ultrasound devices, light energy devices, and the delivery of ablative liquids delivered to, and extracted from, a GI lumen.

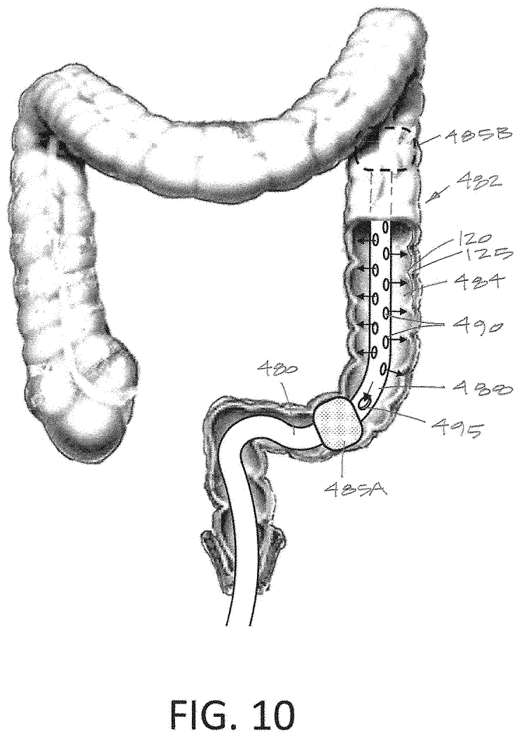

FIG. 10 illustrates another aspect of the invention wherein mechanical means are provided in a catheter 480 for removing and substantially eliminating th residing microbiota and mucous from a targeted region of a GI tract. FIG. 10 illustrates a patient's colon 482 with the distal end of catheter 480 introduced into the lumen 484 thereof. In a variation, the catheter 480 again has a proximal occlusion balloon 485A and a distal occlusion balloon 485B that are expanded in the lumen 484. As can be understood, a large diameter shaft 488 is possible for introducing into a colon, 842 when treating a patient, for example, from 10 to 15 millimeters in diameter or larger. The catheter shaft 488 can be introduced with or without endoscopic vision, and in a variation, an image sensor can be carried at the distal end of the catheter shaft 488. In a large diameter shaft 488, a larger fluid inflow channel with a series of fluid inflow ports 490 can be provided for high rates of fluid inflows adapted for lavage of a targeted region of the lumen 484. Also, one or more outflow ports 495 communicating with a large outflow channel in the shaft 488 can be provided, which is coupled to a negative pressure source. With a catheter of this type, a circulatory flow can be created with high-pressure inflows to wash and lavage the targeted region of the colon 482 to remove and eliminate the microbiota 125 and mucous layer 120 (FIGS. 1-2) from the targeted region. Lavage may further include bactericides, other pharmaceutical agents, or ablative agents such as ethanol to eliminate the residing microbiota 125. Such a lavage may be carried out for one to 5 minutes or more which is believed will remove and eliminate the mucus 120 and microbiota 125 from the targeted region. Thereafter, a restorative microbiota 425 of the types described above can be introduced in a liquid composition through the catheter 480 or by means of suppositories, an implant as described in FIGS. 8-9 above that is tethered in the colon or by orally ingested capsules, tablets, and the like. In general, a method of treating a gastrointestinal (GI) tract dysbiosis comprises eliminating the mucous layers and residing microbiota therein within a targeted region of a GI tract without damaging a submucosal layer of the targeted region and administering a restorative microbiota to the targeted region. The targeted region for such a lavage in a patient can be the colon, esophagus, or small intestine.

Although particular embodiments of the present invention have been described above in detail, it will be understood that this description is merely for purposes of illustration and the above description of the invention is not exhaustive. Specific features of the invention are shown in some drawings and not in others, and this is for convenience only, and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

All references, including publications, patent applications, and patents, cited herein are hereby incorporated by reference to the same extent as if each reference were individually and specifically indicated to be incorporated by reference and were set forth in its entirety herein.

1. A method of treating a gastrointestinal tract dysbiosis, the method comprising:
delivering a condensable vapor to a targeted region of a lumen of a gastrointestinal tract to ablate content of a luminal surface including a mucous layer of the gastrointestinal tract without ablating a submucosal layer of the gastrointestinal tract such that a microbiome of the luminal surface and the mucous layer is ablated.

2. The method of claim 1 further comprising delivering a catheter to the targeted region and expanding a first occlusion balloon and a second occlusion balloon to engage a wall of the gastrointestinal tract, where the first occlusion balloon is spaced apart from the second occlusion balloon, and delivering the condensable vapor through an inflow channel of the catheter to the targeted region between the first occlusion balloon and the second occlusion balloon.

3. The method of claim 2 wherein the condensable vapor undergoes a vapor-to-liquid phase change that applies ablative thermal energy to the luminal surface.

4. The method of claim 3 wherein a portion of a liquid condensate resulting from the vapor-to-liquid phase change flows outward from the targeted region through an outflow channel and an outlet in the catheter.

5. The method of claim 3 wherein the vapor-to-liquid phase change ablates an epithelium of the targeted region.

6. The method of claim 3 wherein the vapor-to-liquid phase change applies ablative thermal energy for 2 to 20 seconds.

7. The method of claim 3 wherein the vapor-to-liquid phase change applies ablative thermal energy at a rate of 5 to 100 cal/second.

8. The method of claim 3 wherein the first occlusion balloon and the second occlusion balloon are spaced apart from 2 cm to 20 cm.

9. The method of claim 3 further comprising ablating the mucous layer of the luminal surface sequentially in a plurality of locations by re-positioning the catheter, the first occlusion balloon and the second occlusion balloon.

10. The method of claim 9 wherein ablating the mucous layer is provided over a continuous length of the gastrointestinal tract of at least 10 cm.

11. The method of claim 9 wherein ablating the mucous layer is within a duodenum.

12. The method of claim 9 wherein ablating the mucous layer is within a jejunum.

13. The method of claim 9 wherein ablating the mucous layer is within all four parts of a duodenum.

14. The method of claim 9 further comprising expanding either the first occlusion balloon or the second occlusion balloon to cover an ampulla of Vater when ablating the luminal surface on either side thereof.

15. The method of claim 1 further comprising administering a restorative microbiota to the targeted region of the gastrointestinal tract in which residing dysbiotic microbiota was ablated.

16. The method of claim 2 further comprising using endoscopic vision during positioning of the catheter positioned and expanding the first occlusion balloon and the second occlusion balloon.

17. The method of claim 2 wherein at least one sensor of at least one occlusion balloon sends a signal of balloon contact with the luminal surface.

18.-51. (canceled)
